(19) United States
(12) Patent Application Publication
(10) Pub. No.: US 2019/0029943 A1
(43) Pub. Date: Jan. 31, 2019

(54) METHODS FOR IMPROVING THE APPEARANCE OF THE SKIN UNDER THE EYE AREA

(71) Applicant: L'OREAL, Paris (FR)

(72) Inventors: Anne-Laure Suzanne BERNARD, New York, NY (US); Alexandra Jane Elisa FARRAN, Dayton, NJ (US)

(73) Assignee: L'OREAL, Paris (FR)

(21) Appl. No.: 15/663,879

(22) Filed: Jul. 31, 2017

(51) Int. Cl. A61K 8/81; A61K 8/58; A61K 8/92; A61Q 19/08

(52) U.S. Cl. CPC A61K 8/8152; A61K 8/8117; A61K 2800/594; A61K 8/92; A61Q 19/08; A61K 8/585

(57) ABSTRACT

The present disclosure relates to methods comprise applying a skin-tightening composition overnight to improve the appearance of the under eye bag area. Compositions comprise at least one thermoplastic elastomer, at least one adhesive polymer, and at least one filler.

### TECHNICAL FIELD

The disclosure relates to methods for improving the appearance of the skin under the eye area.

### BACKGROUND

Skin is primarily comprised of two layers. The outer layer, or epidermis, has a depth of approximately 100 μm. The inner layer, or dermis, has a depth of approximately 3000 μm from the outer surface of the skin and is comprised of a network of fibrous protein known as collagen, which provides skin firmness, and elastin, which supplies skin elasticity and rebound. As a person ages, their skin produces less collagen and elastin each year. As a result, the skin becomes thinner and more fragile with age, and wrinkle formation as a result of aging is inevitable.

In addition, as a person ages, other skin imperfections may appear or become more noticeable. For example, age spots, which are brown or gray sun-induced skin lesions, may appear on sun-exposed skin as a person gets older. It is common for consumers to wish to improve the appearance of such age-related skin imperfections such as wrinkles, crow's feet, age-spots, eye bags, and the like.

While topical cosmetic formulations such as foundation or concealer types of make-up may improve the appearance of some skin imperfections, such formulations are not lasting and cannot reduce the appearance of more pronounced skin imperfections, such as deep wrinkles. Further, while some cosmetic formulations may include an ingredient to reduce the appearance of imperfections over time, such as an anti-wrinkle cream, such formulations may take a long time for results to be noticeable, and may also be ineffective to reduce the appearance of more pronounced skin imperfections.

As an alternate to topical cosmetic formulations, more invasive techniques such as surgery, fillers, or laser resurfacing of the skin may provide longer-lasting effects and can treat prominent imperfections. However, many consumers either cannot afford, or do not wish, to undergo such drastic cosmetic treatments.

As such, there is a consumer desire for topical cosmetic formulations that are effective at reducing the appearance of skin imperfections, such as periorbital puffiness (e.g. under eye bag area).

### SUMMARY OF THE DISCLOSURE

The methods of the instant disclosure provide unexpected results regarding the appearance and reduction of the under eye bag area after applying the composition under the eye area before going to bed.
The method for treating periorbital puffiness of the instant disclosure typically include the following:
(1) forming a film under the eye area skin by applying a skin-tightening composition to the under eye area, the skin-tightening composition comprising:
   a. at least one thermoplastic elastomer chosen from amorphous hydrocarbon block copolymers of styrene and monomers of hydrocarbon containing 2 to 5 carbon atoms and comprising one or two ethylenic unsaturations, and having a first T_{g} below about 0° C., and a second T_{g} greater than about 25° C.;
   b. at least one adhesive film-forming polymer chosen from polymer particles of C₁-C₄ alkyl(methacrylate)polymer, stabilized in a non-aqueous dispersion; and
   c. at least one filler,
      wherein the Young Modulus of the film formed on the under eye area is greater than about 500 kPa;
      wherein the fluid under the eye accumulates substantially less after overnight treatment;
      wherein the skin-tightening composition is applied before bed time.

In one or more embodiments, the disclosure relates to methods wherein the step of applying further comprises applying the skin-tightening composition under the eye area for a duration of at least about 7 hours.

In further embodiments, the disclosure relates to methods for improving the appearance of the skin under the eye area, said methods comprising forming a film on the skin by applying a composition under the eye area, said composition comprising at least one thermoplastic elastomer in the composition in an amount ranging from about 5% to about 25% by weight, relative to the total weight of the composition. In one or more embodiments, the disclosure relates to methods for improving the appearance of the skin under the eye area, said methods comprising forming a film on the skin by applying a composition under the eye area, said composition comprising at least one adhesive polymer chosen from polymer particles comprising about 80% to about 100%, by weight, of C₁-C₄ alkyl (meth)acrylate and of about 0% to about 20%, by weight, of ethylenically unsaturated acid monomer of C₁-C₄ alkyl(methacrylate) polymer in an oil dispersion. In another embodiments, the disclosure relates to methods for improving the appearance of the skin under the eye area, said methods comprising forming a film on the skin by applying a composition under the eye area, said composition comprising the polymer of the particles chosen from:
polymers consisting of at one or more C1-C4 alkyl(methacrylate)polymer; and
polymers consisting essentially of a copolymer of C1-C4 (meth)acrylate and of (meth)acrylic acid or maleic anhydride.

In one or more embodiments, the disclosure relates to methods for improving the appearance of the skin under the eye area, said methods comprising forming a film on the skin by applying a composition under the eye area, said composition contains the C₁-C₄ alkyl(methacrylate)polymer and is chosen from methyl(meth)acrylate, ethyl (meth)acrylate, n-propyl (meth)acrylate, isopropyl (meth)acrylate, n-butyl (meth)acrylate and tert-butyl (meth)acrylate polymers. In further embodiments, the disclosure relates to methods for improving the appearance of the skin under the eye area, said methods comprising forming a film on the skin by applying a composition under the eye area and further comprising at least one additional component chosen from silicone elastomers, humectants, and water. In another embodiment, the disclosure relates to methods for improving the appearance of the skin under the eye area, said methods comprising forming a film on the skin by applying a composition under the eye area, said composition comprising at least one thermoplastic elastomer, at least one adhesive polymer, and at least one filler are present in the skin-tightening composition in a combined amount of greater than about 10% by weight, relative to the weight of the composition. In one embodiment, the disclosure relates to methods for improving the appearance of the skin under the eye area, said methods comprising forming a film on the skin by applying a composition under the eye area, said composition comprising a ratio of thermoplastic elastomer:adhesive polymer in the range of about 1:1 to 8:1. In further embodiments, the disclosure relates to methods for improving the appearance of the skin under the eye area, said methods comprising forming a film on the skin by applying a composition under the eye area, said composition has a consistency G* of greater than about 100 Pa (at 10% strain) and a phase angle below about 45°.

### DETAILED DESCRIPTION

In various embodiments, the disclosure relates to methods for improving the appearance of the skin under the eye area. According to various embodiments, the disclosure relates to compositions comprising at least one thermoplastic elastomer, at least one adhesive polymer, and at least one filler.

The compositions may be effective at reducing the appearance of periorbital puffiness by applying the composition before lying down in bed and for a duration of at least about 7 hours. In various embodiments, the compositions may improve the appearance of the skin by forming a film on the skin that has a Young Modulus greater than that of skin, and thus has the capability of tightening the skin. Additionally, in some embodiments, the film may blur or hide skin imperfections. Accordingly, the disclosure further relates to methods of improving the appearance under the eye area by forming a film on the skin with the compositions described herein.

As used herein, the term “long-lasting” means that the film lasts for at least about 6 hours, such as at least about 12 hours, at least about 24 hours, at least about 48 hours, or at least about 72 hours, after the film is formed on the skin.

As used herein, the term “lasting” it is meant to convey that the film is substantially intact in place on the skin.

As used herein, the term “forms quickly” means that the film forms within less than about 20 minutes, such as less than about 15 minutes, or less than about 10 minutes, after the composition is applied to the skin.

As used herein, the term “blur” with regard to skin imperfections means that the visual appearance of the imperfection is less noticeable.

As used herein, the term “tighten” means that the film contracts in a manner that skin has a tighter feel to the user, and that reduces the visual appearance of the bag under the eye area.

As used herein, the term “soft focus” means that the visual appearance of the skin is more homogenous and matte, leading to the blurring or hiding of skin imperfections.

As used herein, “durable” means the film will not easily rub off, or will not be removed by sweat, water, makeup, lotions, or the like, such that the film will remain substantially intact until removed by the user.

As used herein, “treating” means prevent, reduce the formation of periorbital puffiness due to accumulation of fluid under the eye.

As used herein, the term “periorbital puffiness” or “periorbital edema” also known as “puffy eyes”, or swelling around the eyes, is the appearance of swelling in the tissues around the eyes, called the orbits. It is almost exclusively caused by fluid buildup around the eyes, or periorbital edema.

As used herein, the term “substantially less” means the prevention, the reduction of the fluid accumulation under the eye area.

### Compositions

According to various embodiments, the compositions comprise at least one thermoplastic elastomer, at least one adhesive polymer, and at least one filler, which together form an association. Additional optional components, such as solvents, silicone elastomers, humectants, and water, may also be included in the compositions.

### Thermoplastic Elastomer

According to various exemplary and non-limiting embodiments, the at least one thermoplastic elastomer may be chosen from block copolymers having at least two glass transition temperatures (“T_{g}”). The block copolymers may be hydrocarbon-soluble or dispersible in the oily phase. In various embodiments, the at least one thermoplastic elastomer may be amorphous, crystalline, or semicrystalline.

The block copolymers comprise one or more hard segments attached to one or more soft segments. The hard segments of the thermoplastic elastomer may comprise vinyl monomers in varying amounts. Examples of suitable vinyl monomers include, but are not limited to, styrene, methacrylate, acrylate, vinyl ester, vinyl ether, vinyl acetate, and the like. The soft segments may comprise olefin polymers and/or copolymers which may be saturated, unsaturated, or combinations thereof. Exemplary olefin copolymers may include, but are not limited to, ethylene/propylene copolymers, ethylene/butylene copolymers, propylene/butylene copolymers, polybutylene, polyisoprene, polymers of hydrogenated butanes and isoprenes, and mixtures thereof.

By way of example, the at least one thermoplastic elastomer may be chosen from diblock, triblock, multiblock, radial, and star copolymers obtained by polymerizing at least one unsaturated hydrocarbon monomer having 2 to 5 carbon atoms and having one or two ethylenic unsaturations. Non-limiting examples of unsaturated hydrocarbon monomers having 2 to 5 unsaturated carbon atoms include ethylene, propylene, butadiene, isoprene or pentadiene. In various exemplary and non-limiting embodiments, block copolymers may be chosen from those comprising at least one styrene block and at least one block comprising units selected from butadiene, ethylene, propylene, butylene, isoprene, or mixtures thereof.

Optionally, the block copolymer may be hydrogenated to reduce the residual ethylenic unsaturation after the polymerization of the monomers. For example, the hydrocarbon-based block copolymer may optionally be a hydrogenated copolymer comprising styrene blocks and ethylene blocks/C₃-C₄ alkylene or isoprene blocks. In one exemplary embodiment, the block copolymer is an amorphous hydrocarbon block copolymer, for example an amorphous hydrocarbon block copolymer of styrene and monomers of hydrocarbon containing 2 to 5 carbon atoms and comprising one or two ethylenic unsaturations.

The amorphous thermoplastic elastomers comprise at least one first block whose T_{g} is below about 20° C., such as below about 0° C., below about −20° C., or below about −40° C. The T_{g} of the first block can, for example, range from about −150° C. to about 20° C., such as from about −100° C. to about 0° C. The block copolymers also comprise at least one second block whose T_{g} is greater than about 25° C., such as greater than about 50° C., greater than about 75° C., greater than about 100° C., or greater than about 150° C. The T_{g} of the second block can, for example, range from about 25° C. to about 150° C., such as from about 50° C. to about 125° C., about 60° C. to about 120° C., or about 70° C. to about 100° C.

Exemplary, non-limiting amorphous diblock copolymers may be chosen from styrene-ethylene/propylene copolymers, styrene-ethylene/butadiene copolymers, styrene-ethylene/butylene copolymers, styrene-butadiene, or styrene-isoprene copolymers. Diblock copolymers are sold, for example, under the name Kraton® G1701E by Kraton Polymers.

Exemplary triblock amorphous copolymers may be chosen from styrene-ethylene/propylene-styrene copolymers, styrene-ethylene/butadiene-styrene copolymers, copolymers of styrene-isoprene-styrene, and copolymers of styrene-butadiene-styrene, such as those sold under the names Kraton® G1650, Kraton® D1101, D1102 Kraton®, Kraton® D1160 by Kraton Polymers. In one exemplary embodiment, the thermoplastic elastomer may be a mixture of a triblock copolymer styrene-butylene/ethylene-styrene diblock copolymer and a styrene-ethylene/butylene, such as those sold under the name Kraton® G1657M by Kraton Polymers. In a further example, the thermoplastic elastomer may be a mixture of hydrogenated triblock copolymer styrene-butylene/ethylene-styrene hydrogenated star polymer and ethylene-propylene-styrene, such mixing can in particular be in isododecane in another oil. Such mixtures are sold, for example, by Penreco under the trade names VERSAGEL® M5960 and M5670 VERSAGEL®.

In further exemplary embodiments, the at least one thermoplastic elastomer is chosen from semicrystalline block copolymers having at least two glass transition temperatures. The semicrystalline block copolymers can comprise at least one first block whose T_{g} is greater than about 40° C., such as greater than about 75° C., or greater than 100° C. The T_{g} of the first block can, for example, range from about 40° C. to about 150° C., such as from about 50° C. to about 100° C. The semcrystalline block copolymers also comprise at least one second block whose T_{g} is less than about −50° C., such as less than about −75° C., less than about −100° C., or less than about −150° C. The T_{g} of the second block can, for example, range from about −150° C. to about −50° C., such as from about −100° C. to about −50° C.

By way of non-limiting example, the semicrystalline thermoplastic elastomers may be chosen from copolymers containing a polyamide and/or a polysilicone and/or a polyurethane, for example polysilicone-polyamides or polysilicone-polyurethanes. For example, the semicrystalline thermoplastic elastomers may be chosen from polyorganosiloxane-containing polymers comprising at least one moiety corresponding to formula I:

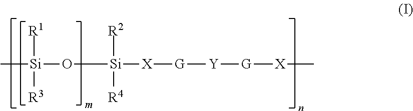

in which:

1) R¹, R², R³ and R⁴, which may be identical or different, represent a group chosen from: (a) linear, branched or cyclic, saturated or unsaturated, C₁ to C₄₀ hydrocarbon-based groups, possibly containing in their chain one or more oxygen, sulphur and/or nitrogen atoms, and possibly being partially or totally substituted with fluorine atoms, (b) C₆ to C₁₀ aryl groups, optionally substituted with one or more C₁ to C₄ alkyl groups, (c) polyorganosiloxane chains possibly containing one or more oxygen, sulphur and/or nitrogen atoms;

2) X, which may be identical or different, represents a linear or branched C₁ to C₃₀ alkylenediyl group, possibly containing in its chain one or more oxygen and/or nitrogen atoms;

3) Y is a saturated or unsaturated, C₁ to C₅₀ linear or branched divalent alkylene, arylene, cycloalkylene, alkylarylene or arylalkylene group, optionally comprising one or more oxygen, sulphur and/or nitrogen atoms, and/or optionally substituted with one of the following atoms or groups of atoms: fluorine, hydroxyl, C₃ to C₈ cycloalkyl, C₁ to C₄₀ alkyl, C₅ to C₁₀ aryl, phenyl optionally substituted with one to three C₁ to C₃ alkyl, C₁ to C₃ hydroxyalkyl, and C₁ to C₆ aminoalkyl groups;

4) G, which may be identical or different, represents a group chosen from ester, amide, sulphonamide, carbamate, thiocarbamate, urea, thiourea groups, and combinations thereof;

5) m is an integer ranging from 1 to 1,000, preferably from 1 to 700 and more preferably from 6 to 200; and

6) n is an integer ranging from 2 to 500 and preferably from 2 to 200.

In further embodiments, the semicrystalline thermoplastic elastomers may be chosen from copolymers containing at least one moiety corresponding to formula II:

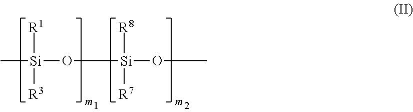

in which:

R¹ and R, which may be identical or different, are as defined above for formula (I),

R⁷ represents a group as defined above for R¹ and R³, or represents a group of formula —X-G-R⁹ in which X and G are as defined above for formula (I) and R⁹ represents a hydrogen atom or a linear, branched or cyclic, saturated or unsaturated, C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,

R⁸ represents a group of formula —X-G-R⁹ in which X, G and R⁹ are as defined above,

m₁ is an integer ranging from 1 to 998, and

m₂ is an integer ranging from 2 to 500.

In yet further embodiments, it is also possible to use a block copolymer comprising several different moieties of formula (I), and/or several different moieties of formula (II), for example a polymer in which at least one of the groups R¹, R², R³, R⁴, X, G, Y, m, and n is different in one of the moieties. It is also possible to use a block copolymer comprising at least one moiety of formula (I) and at least one moiety of formula (II), the moieties of formula (I) and the moieties of formula (II) possibly being identical to, or different from, each other.

For example, in at least one embodiment, the semicrystalline thermoplastic elastomer may be chosen from polyamide copolymers containing at least one moiety corresponding to formula III and at least one moiety corresponding to formula IV:

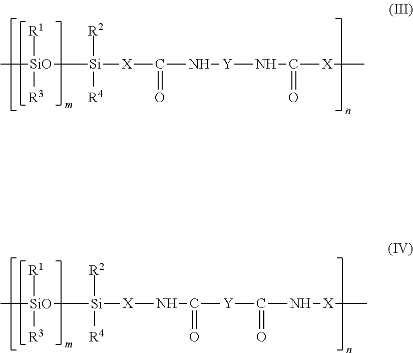

in which:

(a) R¹, R², R³, and R⁴ are the same or different and may be selected from the group consisting of methyl, ethyl, propyl, isopropyl, a siloxane chain, and phenyl;

(b) X is a linear or branched chain alkylene having 1-30 carbons;

(c) Y is selected from the group consisting of linear or branched chain alkylenes having 1-40 carbons;

(d) m is a number between 1 and 700; and

(e) n is a number between 1 and 500.

By way of example only, the semicrystalline thermoplastic elastomer may be chosen from Nylon 6, Nylon 66, and Nylon-611/dimethicone copolymer.

The thermoplastic elastomer may be present in the composition in an amount up to about 25%, such as an amount ranging from about 5% to about 20%, about 6% to about 18%, about 7% to about 16%, about 8% to about 15%, about 9% to about 14%, relative to the weight of the composition.

### Adhesive Polymer

Compositions according to the disclosure further comprise at least one adhesive film-forming polymer. In various embodiments, the at least one adhesive polymer may be amorphous, crystalline, or semicrystalline.

In various embodiments, the adhesive polymer may have a T_{g} greater than about 25° C., such as greater than about 50° C., greater than about 75° C., or greater than about 100° C., according to various embodiments. In further embodiments, the adhesive polymer may have a T_{g} less than about 25° C., such as less than about 0° C., less than about −25° C., or less than about −50° C.

The at least one adhesive polymer may be present in the composition in an amount up to about 25%, such as an amount ranging from about 5% to about 20%, about 6% to about 18%, about 7% to about 16%, about 8% to about 15%, about 9% to about 14%, or relative to the weight of the composition.

As non-limiting examples of adhesive polymers having a T_{g} greater than about 25° C. may be mentioned polymer particles of C₁-C₄ alkyl(methacrylate)polymer, stablilized in a non-aqueous dispersion, referred to herein for ease of reference as an “oil dispersion,” such as those described in WO2015/091513 which is incorporated by reference herein.

By way of example, the C₁-C₄ alkyl (meth)acrylate monomers may be chosen from methyl (meth)acrylate, ethyl (meth)acrylate, n-propyl (meth)acrylate, isopropyl (meth)acrylate, n-butyl (meth)acrylate and tert-butyl (meth)acrylate. For example, the polymer may be a methyl acrylate and/or ethyl acrylate polymer.

The polymer may also comprise an ethylenically unsaturated acid monomer or the anhydride thereof, chosen especially from ethylenically unsaturated acid monomers comprising at least one carboxylic, phosphoric or sulfonic acid function, such as crotonic acid, itaconic acid, fumaric acid, maleic acid, maleic anhydride, styrenesulfonic acid, vinylbenzoic acid, vinylphosphoric acid, acrylic acid, methacrylic acid, acrylamidopropanesulfonic acid or acrylamidoglycolic acid, and salts thereof. For example, the ethylenically unsaturated acid monomer may be chosen from (meth)acrylic acid, maleic acid, and maleic anhydride.

The salts may be chosen from salts of alkali metals, for example sodium or potassium; salts of alkaline-earth metals, for example calcium, magnesium or strontium; metal salts, for example zinc, aluminum, manganese or copper; ammonium salts of formula NH⁺; quaternary ammonium salts; salts of organic amines, for instance salts of methylamine, dimethylamine, trimethylamine, triethylamine, ethylamine, 2-hydroxyethylamine, bis(2-hydroxyethyl)amine or tris(2-hydroxyethyl)amine; lysine or arginine salts.

The polymer of the particles of the oil dispersion may thus comprise or consist essentially of about 80% to about 100%, by weight, of C₁-C₄ alkyl (meth)acrylate and of about 0% to about 20%, by weight, of ethylenically unsaturated acid monomer, relative to the total weight of the polymer. According to one exemplary embodiment, the polymer consists essentially of a polymer of one or more C₁-C₄ alkyl (meth)acrylate monomers. According to another exemplary embodiment, the polymer consists essentially of a copolymer of C₁-C₄ (meth)acrylate and of (meth)acrylic acid or maleic anhydride.

By way of non-limiting example only, the polymer of the particles in the oil dispersion, which may optionally be crosslinked or alternatively may not be crosslinked, may be chosen from methyl acrylate homopolymers, ethyl acrylate homopolymers, methyl acrylate/ethyl acrylate copolymers, methyl acrylate/ethyl acrylate/acrylic acid copolymers, methyl acrylate/ethyl acrylate/maleic anhydride copolymers, methyl acrylate/acrylic acid copolymers, ethyl acrylate/acrylic acid copolymers, methyl acrylate/maleic anhydride copolymers, and ethyl acrylate/maleic anhydride copolymers.

The polymer of the particles in the dispersion may have a number-average molecular weight ranging from about 2000 to about 10,000,000, for example ranging from about 150,000 to about 500,000. The polymer particles may be present in the oil dispersion in a content ranging from about 20% to about 60%, for example about 21% to about 58.5%, about 30% to about 50%, about 35% to about 45%, or about 36% to about 42%, by weight, relative to the total weight of the oil dispersion.

The stabilizer in the oil dispersion may be an isobornyl (meth)acrylate polymer chosen from isobornyl (meth)acrylate homopolymer and statistical copolymers of isobornyl (meth)acrylate and of C₁-C₄ alkyl (meth)acrylate present in an isobornyl (meth)acrylate/C₁-C₄ alkyl (meth)acrylate weight ratio of greater than about 4, for example greater than about 4.5, or greater than about 5. For example, the weight ratio may range from about 4.5 to about 19, such as from about 5 to about 19, or from about 5 to about 12.

By way of example only, the stabilizer may be chosen from isobornyl acrylate homopolymers, statistical copolymers of isobornyl acrylate/methyl acrylate, statistical copolymers of isobornyl acrylate/methyl acrylate/ethyl acrylate, and statistical copolymers of isobornyl methacrylate/methyl acrylate.

In various embodiments, the stabilizer may have a number-average molecular weight ranging from about 10,000 to about 400,000, such as from about 20,000 to about 200,000.

In various embodiments, the combination of the stabilizer+polymer of the particles present in the oil dispersion comprises from about 10% to about 50%, such as about 15% to about 30%, by weight of polymerized isobornyl (meth)acrylate, and from about 50% to about 90%, such as about 70% to about 85%, by weight of polymerized C₁-C₄ alkyl (meth)acrylate, relative to the total weight of the combination of the stabilizer+polymer of the particles.

The oily medium of the oil dispersion comprises a hydrocarbon-based oil. The hydrocarbon-based oil is an oil that is liquid at room temperature (25° C.). The term “hydrocarbon-based oil” means an oil formed essentially from, or even consisting of, carbon and hydrogen atoms, and optionally oxygen and nitrogen atoms, and not containing any silicon or fluorine atoms. It may contain alcohol, ester, ether, carboxylic acid, amine and/or amide groups.

Exemplary and non-limiting embodiments of the hydrocarbon-based oil medium of the oil dispersion include hydrocarbon-based oils containing up to about 40, such as from 8 to 16 or from 8 to 14, carbon atoms. Optionally, the hydrocarbon-based oil is apolar. For example, the hydrocarbon based oil may be chosen from isododecane.

The oil dispersion may be prepared, for example, as described in WO2015/091513.

Alternatively, the adhesive polymer may be chosen from aliphatic or cycloaliphatic hydrocarbon polymers selected from aliphatic or cycloaliphatic hydrocarbon resins having a T_{g} greater than about 25° C. By “aliphatic or cycloaliphatic hydrocarbon resins,” it is meant polymers or copolymers of olefins or polymers or copolymers of partly or totally hydrogenated aromatic hydrocarbon monomers. For example, the adhesive polymer may be chosen from aliphatic hydrocarbon resins, aromatic modified aliphatic hydrocarbon resins, hydrogenated polycyclopentadiene resins, polycyclopentadiene resins, gum rosins, gum rosin esters, wood rosins, wood rosin esters, tall oil rosins, tall oil rosin esters, polyterpenes, aromatic modified polyterpenes, terpene phenolics, aromatic modified hydrogenated polycyclopentadiene resins, hydrogenated aliphatic resin, hydrogenated aliphatic aromatic resins, hydrogenated terpenes and modified terpenes, hydrogenated rosin acids, hydrogenated rosin esters, polyisoprene, partially or fully hydrogenated polyisoprene, polybutenediene, partially or fully hydrogenated polybutenediene, and hydrogenated styrene/methyl styrene/indene copolymers. In various embodiments, hydrogenated indene/methylstyrene/styrene copolymers marketed under the name of REGALITE® by Eastman Chemical, may be chosen. For example, REGALITE® R1090, REGALITE® R1100, REGALITE® S1100, REGALITE® R7100, REGALITE® R1010, REGALITE® R112, or REGALITE® S5100 may be chosen. As further examples, those sold under the name of ARKON® P-90, ARKON® P-100, and ARKON® P-115, by Arakawa, may be chosen.

In further embodiments, the adhesive polymer may have a T_{g} of less than about 25° C. For example, the at least one adhesive polymer may be chosen from polyacids, such as hyperbranched polyacids. Polyacids useful according to various embodiments of the disclosure may be found in U.S. Pat. No. 7,582,719 and US2013/0236409, both of which are incorporated by reference herein.

The term “hyperbranched polyacid” refers to the fact that the functional groups of the hyperbranched functional polymer are substituted with carboxylic acid groups. Unsaturated functionalizing compounds useful include, but are not limited to, carboxylic acids, carboxylic acid esters, amides, ethers, amines, phosphate esters, silanes and alcohols. Examples of such carboxylic acids include, but are not limited to, 5-hexenoic acid, 6-heptenoic acid, 10-undecylenic acid, 9-decenoic acid, oleic acid, and erucic acid. Also useful are esters of these acids with linear or branched-chain alcohols having from about 1 to about 10 carbon atoms, as well as triglycerides containing olefinic unsaturation in the fatty acid portion such as tall oil, fish oils, soybean oil, linseed oil, cottonseed oil and partially hydrogenated products of such oils. Other useful materials include olefinic alcohols such as allyl alcohol, 9-decen-1-ol, 10-undecylenyl alcohol, oleyl alcohol, erucyl alcohol, acetic acid or formic acid esters of these alcohols, C1-C4 alkyl ether derivatives of these alcohols and formamides or acetamides of unsaturated amines such as oleylamine, erucylamine, 10-undecylenylamine and allylamine.

In various embodiments, the hyperbranched polyacid compound useful according to the disclosure may have at least two carboxyl groups. In various embodiments, the hyperbranched polyacid has a carboxyl number of at least 3, such as at least 10, at least 50, at least 100, or at least about 150. According to various embodiments, the hyperbranched polyacid has a carboxyl number ranging from about 50 to about 250, such as ranging from about 75 to about 225, about 100 to about 200, or about 125 to 175. In one embodiment, the hyperbranched polyacid has a carboxyl number ranging from 90 to 150.

In various embodiments, the at least one hyperbranched acid compound has a molecular weight (Mw) ranging from about 500 to about 25,000, such as ranging from about 800 to about 10,000, or from about 1000 to about 8000. In one embodiment, the hyperbranched polyacid has a Mw ranging from about 1000 to about 6000.

In various embodiments, the at least one hyperbranched polyacid compound has a viscosity at 210° F. ranging from 0.01 Pas to 10 Pas, such as from 0.02 to 7 Pas, or from 0.03 to 6 Pas, including all ranges and subranges there between. The viscosity is determined using Brookfield viscometer at 210° F. by ASTMD-3236MOD method. In various embodiments, the at least one hyperbranched acid compound has an acid number ranging from about 20 to about 400 mg/KOH, such as from about 30 to about 300 mg/KOH, or ranging from about 50 to about 100 mg/KOH.

In one exemplary embodiment, the at least one adhesive polymer is a polyacid chosen from C₃₀₊ olefin/undecylenic acid copolymers, such as C₂₈-C₅₂ olefin/undecylenic acid copolymers, for example those available from New Phase Technologies under trade name Performa V6112™.

As yet further examples of adhesive polymers that may be chosen are acrylic type film formers. As used herein, “acrylic type film formers” include polymers that are film forming agents and which are based upon one or more (meth)acrylic acid (and corresponding (meth)acrylate) monomers or similar monomers.

Non-limiting examples of such film forming agents include copolymers containing at least one apolar monomer, at least one olefinically unsaturated monomer, and at least one vinylically functionalized monomer.

For the apolar monomers, acrylic monomers which comprise acrylic and methacrylic esters with alkyl groups composed of 4 to 14 C atoms, preferably 4 to 9 C atoms may be chosen. Examples of monomers of this kind include n-butyl acrylate, n-butyl methacrylate, n-pentyl acrylate, n-pentyl methacrylate, n-amyl acrylate, n-hexyl acrylate, hexyl methacrylate, n-heptyl acrylate, n-octyl acrylate, n-octyl methacrylate, n-nonyl acrylate, isobutyl acrylate, isooctyl acrylate, isooctyl methacrylate, and their branched isomers, such as, for example, 2-ethylhexyl acrylate, 2-ethylhexyl methacrylate.

For olefinically unsaturated monomers, it is possible to use monomers having functional groups selected from hydroxyl, carboxyl, sulphonic acid groups, phosphonic acid groups, acid anhydrides, epoxides, and amines. Examples of olefinically unsaturated monomers include acrylic acid, methacrylic acid, itaconic acid, maleic acid, fumaric acid, crotonic acid, aconitic acid, dimethylacrylic acid, beta-acryloyloxypropionic acid, trichloracrylic acid, vinylacetic acid, vinylphosphonic acid, itaconic acid, maleic anhydride, hydroxyethyl acrylate, hydroxypropyl acrylate, hydroxyethyl methacrylate, hydroxypropyl methacrylate, 6-hydroxyhexyl methacrylate, allyl alcohol, glycidyl acrylate, glycidyl methacrylate.

For vinylically functionalized compounds, exemplary monomers include monomers which are copolymerizable with one or both of the previously discussed monomers and include, for example, methyl acrylate, ethyl acrylate, propyl acrylate, methyl methacrylate, ethyl methacrylate, benzyl acrylate, benzyl methacrylate, sec-butyl acrylate, tert-butyl acrylate, phenyl acrylate, phenyl methacrylate, isobornyl acrylate, isobornyl methacrylate, tert-butylphenyl acrylate, tert-butylphenyl methacrylate, dodecyl methacrylate, isodecyl acrylate, lauryl acrylate, n-undecyl acrylate, stearyl acrylate, tridecyl acrylate, behenyl acrylate, cyclohexyl methacrylate, cyclopentyl methacrylate, phenoxyethyl acrylate, phenoxyethyl methacrylate, 2-butoxyethyl methacrylate, 2-butoxyethyl acrylate, 3,3,5-trimethylcyclohexyl acrylate, 3,5-di methyladamantyl acrylate, 4-cumylphenyl methacrylate, cyanoethyl acrylate, cyanoethyl methacrylate, 4-biphenyl acrylate, 4-biphenyl methacrylate, 2-naphthyl acrylate, 2-naphthyl methacrylate, tetrahydrofurfuryl acrylate, diethylaminoethyl acrylate, diethylaminoethyl methacrylate, dimethylaminoethyl acrylate, dimethylaminoethyl methacrylate, 2-butoxyethyl acrylate, 2-butoxyethyl methacrylate, methyl 3-methoxyacrylate, 3-methoxybutyl acrylate, phenoxyethyl acrylate, phenoxyethyl methacrylate, 2-phenoxyethyl methacrylate, butyldiglycol methacrylate, ethylene glycol acrylate, ethylene glycol monomethylacrylate, methoxy-polyethylene glycol methacrylate 350, methoxy-polyethylene glycol methacrylate 500, propylene glycol monomethacrylate, butoxydiethylene glycol methacrylate, ethoxytriethylene glycol methacrylate, octafluoropentyl acrylate, octafluoropentyl methacrylate, 2,2,2-trifluoroethyl methacrylate, 1,1,1,3,3,3-hexafluoroisopropyl acrylate, 1,1,1,3,3,3-hexafluoroisopropyl methacrylate, 2,2,3,3,3-pentafluoropropyl methacrylate, 2,2,3,4,4,4-hexafluorobutyl methacrylate, 2,2,3,3,4,4,4-heptafluorobutyl acrylate, 2,2,3,3,4,4,4-heptafluorobutyl methacrylate, 2,2,3,3,4,4,5,5,6,6,7,7,8,8,8-pentadecafluorooctyl methacrylate, dimethylaminopropylacrylamide, dimethylaminopropylmethacrylamide, N-(1-methylundecyl)acrylamide, N-(n-butoxymethyl)acrylamide, N-(butoxymethyl)methacrylamide, N-(ethoxymethyl)acrylamide, N-(n-octadecyl)acrylamide, and also N,N-dialkyl-substituted amides, such as, for example, N,N-dimethylacrylamide, N,N-dimethylmethacrylamide, N-benzylacrylamides, N-isopropylacrylamide, N-tert-butylacrylamide, N-tert-octylacrylamide, N-methylolacrylamide, N-methylolmethacrylamide, acrylonitrile, methacrylonitrile, vinyl ethers, such as vinyl methyl ether, ethyl vinyl ether, vinyl isobutyl ether, vinyl esters, such as vinyl acetate, vinyl chloride, vinyl halides, vinylidene chloride, vinylidene halide, vinylpyridine, 4-vinylpyridine, N-vinylphthalimide, N-vinyllactam, N-vinylpyrrolidone, styrene, a- and p-methylstyrene, a-butylstyrene, 4-n-butylstyrene, 4-n-decylstyrene, 3,4-dimethoxystyrene, macromonomers such as 2-polystyrene-ethyl methacrylate (molecular weight, Mw, of 4000 to 13 000 g/mol), poly(methyl methacrylate)ethyl methacrylate (Mw of 2000 to 8000 g/mol).

As exemplary acrylic type film formers, mention may be made of copolymers of acrylic acid, isobutyl acrylate and isobornyl acetate, such as that sold under the names Pseudoblock (Chimex) and Synamer-3. In both of these commercial products, the copolymer is present with a solvent in a 1:1 ratio (50% solid). Another exemplary film former is Poly(isobornyl methacrylate-8 co-isobornyl acrylate-co-isobutyl acrylate-co-acrylic acid) at 50% of active material in 50% of octyldodecyl neopentanoate (Mexomere PAZ from Chimex).

### Fillers

The compositions comprise at least one filler. The fillers may be mineral or organic in nature, and of any shape. In various embodiments, the fillers may have a particle size greater than about 100nm, and/or a specific surface area greater than about 200 m²/g.

By way of non-limiting example, fillers may be chosen from talc, mica, silica, silica surface-treated with a hydrophobic agent, fumed silica, kaolin, polyamide (Nylon®) powders (e.g. Orgasol® from Atochem), polyurethane powders, poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie) or of acrylic acid copolymers (Polytrap® from the company Dow Corning) and silicone resin microbeads (Tospearls® from Toshiba, for example), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc stearate, magnesium stearate or lithium stearate, zinc laurate or magnesium myristate.

In at least certain embodiments, the at least one filler may be chosen from hydrophobic silica aerogel particles. Silica aerogels are porous materials obtained by replacing (by drying) the liquid component of a silica gel with air.

They are generally synthesized via a sol-gel process in liquid medium and then dried, usually by extraction of a supercritical fluid, the one most commonly used being supercritical CO₂. This type of drying makes it possible to avoid shrinkage of the pores and of the material. The sol-gel process and the various drying processes are described in detail in Brinker C J., and Scherer G. W., Sol-Gel Science: New York: Academic Press, 1990.

The hydrophobic silica aerogel particles used in the present invention have a specific surface area per unit of mass (S_{M}) ranging from 500 to 1500 m²/g, preferably from 600 to 1200 m²/g and better still from 600 to 800 m²/g, and a size expressed as the mean volume diameter (D[0.5]), ranging from 1 to 30 μm, preferably from 5 to 25 μm, better still from 5 to 20 μm and even better still from 5 to 15 μm.

The specific surface area per unit of mass may be determined via the BET (Brunauer-Emmett-Teller) nitrogen absorption method described in the Journal of the American Chemical Society, vol. 60, page 309, February 1938 and corresponding to the international standard ISO 5794/1 (appendix D). The BET specific surface area corresponds to the total specific surface area of the particles under consideration.

The size of the silica aerogel particles may be measured by static light scattering using a commercial granulometer such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is especially described in the publication by Van de Hulst, H. C., “Light Scattering by Small Particles,” Chapters 9 and 10, Wiley, New York, 1957.

The silica aerogel particles used in the present invention may advantageously have a tamped density r) ranging from 0.04 g/cm³ to 0.10 g/cm³ and preferably from 0.05 g/cm³ to 0.08 g/cm³.

In the context of the present invention, this density, known as the tamped density, may be assessed according to the following protocol:

40 g of powder are poured into a measuring cylinder; the measuring cylinder is then placed on a Stay 2003 machine from Stampf Volumeter; the measuring cylinder is then subjected to a series of 2500 packing motions (this operation is repeated until the difference in volume between two consecutive tests is less than 2%); the final volume Vf of packed powder is then measured directly on the measuring cylinder. The tamped density is determined by the ratio m/Vf, in this instance 40/Vf (Vf being expressed in cm³ and m in g).

According to one embodiment, the hydrophobic silica aerogel particles used in the present invention have a specific surface area per unit of volume S_{V} ranging from 5 to 60 m²/cm³, preferably from 10 to 50 m²/cm³ and better still from 15 to 40 m²/cm³.

The specific surface area per unit of volume is given by the relationship:

S_{V}=S_{M}·r where r is the tamped density expressed in g/cm³ and S_{M} is the specific surface area per unit of mass expressed in m²/g, as defined above.

Preferably, the hydrophobic silica aerogel particles according to the invention have an oil-absorbing capacity, measured at the wet point, ranging from 5 to 18 mL/g, preferably from 6 to 15 mL/g and better still from 8 to 12 mL/g.

The oil-absorbing capacity measured at the wet point, noted Wp, corresponds to the amount of water that needs to be added to 100 g of particle in order to obtain a homogeneous paste.

It is measured according to the wet point method or the method for determining the oil uptake of a powder described in standard NF T 30-022. It corresponds to the amount of oil adsorbed onto the available surface of the powder and/or absorbed by the powder by measuring the wet point, described below:

An amount m=2 g of powder is placed on a glass plate, and the oil (isononyl isononanoate) is then added dropwise. After addition of 4 to 5 drops of oil to the powder, mixing is performed using a spatula, and addition of oil is continued until a conglomerate of oil and powder has formed. At this point, the oil is added one drop at a time and the mixture is then triturated with the spatula. The addition of oil is stopped when a firm, smooth paste is obtained. This paste must be able to be spread on the glass plate without cracking or forming lumps. The volume Vs (expressed in mL) of oil used is then noted. The oil uptake corresponds to the ratio Vs/m.

The aerogels used according to the present invention are hydrophobic silica aerogels, preferably of silylated silica (INCI name: silica silylate).

The term “hydrophobic silica” means any silica whose surface is treated with silylating agents, for example halogenated silanes such as alkylchlorosilanes, siloxanes, in particular dimethylsiloxanes such as hexamethyldisiloxane, or silazanes, so as to functionalize the OH groups with silyl groups Si—Rn, for example trimethylsilyl groups.

As regards the preparation of hydrophobic silica aerogels particles that have been surface-modified by silylation, reference may be made to document U.S. Pat. No. 7,470,725.

Use will be made in particular of hydrophobic silica aerogels particles surface-modified with trimethylsilyl groups.

As hydrophobic silica aerogels that may be used in the invention, examples that may be mentioned include the aerogel sold under the name VM-2260 (INCI name: Silica silylate), by the company Dow Corning, the particles of which have a mean size of about 1000 μm and a specific surface area per unit of mass ranging from 600 to 800 m²/g.

In other embodiments, the aerogels sold by the company Cabot under the names Aerogel TLD 201®, Aerogel OGD 201®, and Aerogel TLD 203®, CAB-O-SIL TS-530, CAB-O-SIL TS-610, CAB-O-SIL TS-720, Enova Aerogel MT 1100®, and Enova Aerogel MT 1200®, may be chosen.

Use will be made more particularly of the aerogel sold under the name VM-2270 (INCI name: Silica silylate), by the company Dow Corning, the particles of which have a mean size ranging from 5-15 μm and a specific surface area per unit of mass ranging from 600 to 800 m²/g. It has an oil absorption capability of 1090 mL/100 g based on isononyl isononanoate.

Optionally, mixtures of fillers may be present in the compositions according to the disclosure. For example, a mixture of different aerogel particles, or of an aerogel and a different type of filler, may be used.

The at least one filler may be present in a total amount ranging from about 0.1% to about 20% by weight, for example from about 0.2% to about 15%, from about 0.5% to about 10%, or from about 1% to about 6%, by weight, relative to the total weight of the composition. In at least certain exemplary embodiments, the filler is present in an amount less than about 5%, such as less than about 4%, by weight, relative to the total weight of the composition. In one embodiment, the filler is present in an amount up to about 3% by weight, relative to the total weight of the composition.

### Additional Components

The compositions according to the disclosure may optionally further comprise additional components, such as solvents, silicone elastomers, humectants, and water.

Solvents

The compositions may comprise at least one solvent. Optionally, the compositions may comprise at least one solvent chosen from solvents having a vapor pressure at room temperature (25° C.) of greater than about 100 Pa, such as greater than about 500 Pa, or greater than about 1000 Pa. In various embodiments, the composition is free or substantially free of solvents having a vapor pressure at room temperature (25° C.) of less than about 25 Pa. In further embodiments, the composition may comprise at least one solvent having a vapor pressure at room temperature (25° C.) of greater than about 100 Pa, such as greater than 500 Pa, or greater than 1000 Pa, and at least one solvent having a vapor pressure at room temperature (25° C.) of less than about 100 Pa, such as less than about 50 Pa, or less than about 25 Pa.

In various embodiments, the compositions comprise at least one volatile organic solvent. The volatile organic solvent may be chosen from, for example, volatile hydrocarbon-based oils and volatile silicone oils.

For example, volatile hydrocarbon oils include, but are not limited to, those having from 8 to 16 carbon atoms and their mixtures, such as branched C₈ to C₁₆ alkanes and C₈ to C₁₆ isoalkanes (also known as isoparaffins), isododecane, isodecane, isohexadecane. For example, the at least one solvent may be chosen from the oils sold under the trade names of Isopar® or Permethyl®, the C₈ to C₁₆ branched esters such as isohexyl or isodecyl neopentanoate and their mixtures. In at least certain embodiments, the volatile hydrocarbon oils have a flash point of at least 40° C. It is also possible to use mixtures of isoparaffins and other volatile hydrocarbon-based oils, such as petroleum distillates.

Further, volatile silicone oils may be chosen from linear or cyclic silicone oils, such as those having a viscosity at room temperature (25° C.) of less than or equal to 6 cSt and having from 2 to 7 silicon atoms, these silicones being optionally substituted with alkyl or alkoxy groups of 1 to 10 carbon atoms. Examples of volatile silicone oils that may be used include, but are not limited to, octamethyltetrasiloxane, decamethylcyclo-pentasiloxane, dodecamethylcyclohexasiloxane, heptamethyloctyltrisiloxane, hexamethyldisiloxane, decamethyltetrasiloxane, dodecamethylpentasiloxane, and their mixtures. In at least certain embodiments, the volatile silicone oils have a flash point of at least 40° C.

Additionally, the at least one volatile solvent may be chosen from polar volatile solvents, including but are not limited to, alcohols, volatile esters and volatile ethers.

The at least one solvent may be present in the composition in an amount up to about 95%, such as up to about 90%, up to about 85%, up to about 80%, up to about 75%, up to about 70%, up to about 65%, up to about 60%, up to about 55%, or up to about 50%, by weight of the composition. For example, the at least one solvent may be present in the composition in an amount ranging from about 40% to about 95%, such as about 50% to about 90%, or about 60% to about 85%, or about 65% to about 80%.

Silicone Elastomer

The composition may further optionally comprise at least one silicone elastomer. Surprisingly, in certain embodiments, the at least one silicone elastomer may improve properties such as the thickness and water-resistance of the film, without significantly affecting the mechanical or optical properties of the film. In other embodiments, the addition of at least one silicone elastomer may decrease wettability by sebum, which will help prevent the film from losing tightening properties. It may, in at least certain embodiments, be advantageous to choose a silicone elastomer having greater than 1% active material (AM), such as greater than 2% AM.

The at least one silicone elastomer may, for example, be chosen from at least one silicone crosspolymer dispersed in at least one oil. The at least one silicone crosspolymer may, in certain embodiments, be chosen from dimethicone crosspolymers, such as dimethicone/vinyl dimethicone crosspolymers and dimethicone/phenyl vinyl dimethicone crosspolymers. In other embodiments, the silicone cross-polymer may be modified by one or more groups chosen from alkyl, polyether, polyglycerin groups. For instance, the alkyl modified silicone cross-polymers may be chosen from vinyl dimethicone/lauryl dimethicone cross-polymers, cetearyl dimethicone cross-polymers, and C₃₀-C₄₅ alkyl cetearyl dimethicone cross-polymers. Non-limiting examples of polyether modified silicone cross-polymers include dimethicone/PEG-10/15 cross-polymers. Exemplary alkyl and polyether modified silicone cross-polymers may be chosen, for example, from PEG-10/lauryl dimethicone cross-polymers and PEG-15/lauryl dimethicone cross-polymers. Exemplary polyglycerin modified silicone cross-polymers include dimethicone/polyglycerin-3 cross-polymers and lauryl dimethicone/polyglycerin-3 cross-polymers.

In at least certain embodiments, the silicone polymers do not comprise polyethylene glycol or polypropylene groups, or hydrophilic moieties. Optionally, the silicone elastomer may be chosen from the silicone organic blends isododecane (and) dimethicone crosspolymer (18% AM) sold under the name EL-8040 ID or dimethicone/bis-isobutyl PPG-20 crosspolymer (17% AM in isododecane) sold under the name EL-8050 ID, by Dow Corning; or isododecane (and) vinyldimethyl/trimethylsiloxysilicate stearyl dimethicone crosspolymer (20% AM in isododecane), sold under the name GEL BELSIL RG90 by Wacker.

The silicone crosspolymer may be dispersed in at least one oil. In certain embodiments, the oil may be chosen from silicone oils, such as cyclic and linear organopolysiloxanes. Cyclic organopolysiloxanes may include, for example, cyclotetrasiloxane; cyclopentasiloxane; and methylated cyclic organopolysiloxanes, for example, octamethylcyclotetrasiloxane and decamethylcyclopentasiloxane. Non-limiting examples of linear organopolysiloxanes include low molecular weight dimethicones; high molecular weight dimethicones; alkyl derivatives of linear organopolysiloxanes, for example, cetyl dimethicone and lauryl trimethicone; aryl derivatives of linear organopolysiloxanes, for example, phenyl trimethicone; and hydroxylated derivatives of linear organopolysiloxanes, for example, dimethiconol. In other embodiments, the oil may be chosen from organic oils, such as mineral oil; linear and branched alkanes, for example, isododecane; triethylhexanoin; and squalane.

The at least one silicone crosspolymer may, in some embodiments, comprise from about 5% to about 35% by weight, relative to the total weight of the silicone elastomer blend, for example, from about 10% to about 20% by weight, or from about 25% to about 35% by weight, or from about 20% to about 30% by weight. The at least one oil may comprise from about 65% to about 95% by weight, relative to the total weight of the silicone elastomer blend, such as from about 80% to about 90% by weight, or from about 65% to about 75% by weight, or from about 70% to about 80% by weight.

In various exemplary embodiments, the silicone elastomer blend comprises from about 20% to about 30% of dimethicone/vinyl dimethicone cross-polymer. In further exemplary embodiments, the silicone elastomer blend comprises from about 70% to about 80% by weight of dimethicone. In yet further exemplary embodiments, the silicone elastomer blend comprises from about 20% to about 30% of dimethicone/vinyl dimethicone cross-polymer and from about 70% to about 80% by weight dimethicone.

For example, silicone elastomers sold under the name KSG-16 dimethicone (and) dimethicone/vinyl dimethicone corpsspolymer, KSG-21 (at 27% in active material) INCI name: Dimethicone/PEG-10 Dimethicone vinyl dimethicone crosspolymer), KSG-20 (at 95% % in active material) INCI name: PEG-10 Dimethicone Crosspolymer), KSG-30, (at 100% % in active material) INCI name: Lauryl PEG-15 Dimethicone vinyl dimethicone crosspolymer), KSG-31 (at 25% in active material) INCI name: Lauryl PEG-15 Dimethicone vinyl dimethicone crosspolymer), KSG-32 or KSG-42 or KSG-320 or KSG-30 (at 25% in active material) INCI name: Lauryl PEG-15 Dimethicone vinyl dimethicone crosspolymer), KSG-33: Lauryl PEG-15 (at 20% in active material) Dimethicone vinyl dimethicone crosspolymer), KSG-210 (at 25% in active material) INCI name: Dimethicone/PEG-10/15 crosspolymer), KSG-310: lauryl modified polydimethylsiloxane polyoxyethylenated in mineral oil, KSG-330 and KSG-340: PEG-15/lauryl dimethicone crosspolymer, and X-226146 (at 32% % in active material) INCI name: Dimethicone/PEG-10 Dimethicone vinyl dimethicone crosspolymer), all by Shin Etsu; DC9010 (at 9% in active material) and DC9011 (at 11% in active material) INCI name: PEG-12 dimethicone crosspolymer), DC9040 cyclopentasiloxane (and) dimethicone crosspolymer, and DC9041 dimethicone (and) dimethicone crosspolymer, all by Dow Corning; or the products sold under the VELVESIL product line by Momentive, such as VELVESIL 125 and VELVESIL DM, may be chosen.

Other examples of silicone elastomers include KSG-710 (at 25% in active material, INCI name: dimethicone/polyglycerin-3 crosspolymer); and KSG-820, KSG-830 and KSG-840, all of which are dimethicone/polvaleverin-3 crosspolymer (INCI), but in different diluents, 820 is in isododecane, 830 is in triethyl hexanoin, and 840 is in squalene, all by Shin Estu.

The at least one silicone elastomer may optionally be included in the composition in an amount up to about 10%, such as up to about 8%, up to about 5%, about 4.5%, up to about 4%, up to about 3.5%, up to about 3%, up to about 2.5%, up to about 2%, up to about 1.5%, up to about 1%, up to about 0.75%, up to about 0.5%, up to about 0.25%, up to about 0.2%, or up to about 0.1%, by weight, relative to the weight of the composition. In certain embodiments, the at least one silicone elastomer may be present in an amount ranging from about 1% to about 10%, such as about 2% to about 8%, about 3% to about 6%, or about 4% to about 5%, by weight, relative to the weight of the composition.

Humectants

Optionally, compositions according to the disclosure may comprise at least one humectant or moisturizing agent. Surprisingly, in at least certain embodiments, the at least one humectant may improve the optical properties and feeling of the film formed on the skin by the composition, without negatively affecting the mechanical properties of the film.

By way of example only, humectants or moisturizing agents may be chosen from polyhydroxy compounds including but not limited to glycerin and glycols such as, for example, propylene glycol, butylene glycol, dipropylene glycol and diethylene glycol, glycol ethers such as monopropylene, dipropylene and tripropylene glycol alkyl(C₁-C₄)ethers, monoethylene, diethylene and triethylene glycol.

The at least one humectant may be present in the composition in an amount up to about 20%, such as up to about 15%, up to about 14%, up to about 13%, up to about 12%, up to about 11%, up to about 10%, up to about 9%, up to about 8%, up to about 7%, up to about 6%, up to about 5%, up to about 4%, up to about 3%, up to about 2%, up to about 1%, or up to about 0.5%, by weight of the composition.

Water

Optionally, in at least certain embodiments, water may be added to the compositions according to the disclosure. Surprisingly, in certain non-limiting embodiments, water may improve the properties of the film formed on the skin by the composition, such as Young Modulus, transparency, cohesion, and thickness.

Water can be included in the composition in an amount up to about 15%, up to about 12%, up to about 10%, up to about 9%, up to about 8%, up to about 7%, up to about 6%, up to about 5%, up to about 4%, up to about 3%, up to about 2%, up to about 1%, or up to about 0.5%, by weight of the composition. In at least certain embodiments, the compositions are anhydrous or substantially anhydrous. In other embodiments, the compositions may be in the form of a water-in-oil (W/O) emulsion.

It may, in at least certain embodiments, be advantageous to include water and at least one humectant, for example water and glycerin, in the composition together.

### Film

When the compositions according to the disclosure are applied to the skin, the at least one thermoplastic elastomer, the at least one adhesive polymer, and the at least one filler together form a matrix that creates a film on the skin. The films formed by the compositions described herein form quickly, are long-lasting and durable, and have optical properties that are advantageous for a skin-tightening film, such as transparency, matte effect, and a soft focus effect which helps to blur skin imperfections so that they are less noticeable.

Additionally, as discussed above, the compositions according to the disclosure form a film that is stiffer than, and thus capable of tightening, human skin. Human skin has a Young Modulus in the range of 10 kPa to 100 kPa; thus, a film for tightening the skin should have a Young Modulus of greater than 100 kPa. The films that are formed by the compositions have Young Modulus' greater than 500 kPa (0.5 MPa) in some embodiments, greater than 1000 kPa (1 MPa) in some embodiments, greater than 5000 kPa (5 MPa) in some embodiments, and even greater than 10,000 kPa (10 MPa) in some embodiments. Additionally, the compositions according to the disclosure have sufficient consistency G* and phase angle below 45°, in order to form an effective and lasting film on the skin.

As such, the amounts and components of the composition should be chosen to provide a film on the skin that is capable of tightening the skin, while also blurring skin imperfections.

In various exemplary embodiments, for the best film properties, it may be advantageous for the total amount of thermoplastic elastomer plus adhesive polymer plus filler to be greater than about 10%, such as greater than about 15% or greater than about 20%, by weight, of the total weight of the composition.

In yet further exemplary embodiments, for the best film properties, it may be advantageous for amounts of the thermoplastic elastomer and adhesive polymer to be chosen so that the ratio of thermoplastic elastomer:adhesive polymer is in the range of about 1:10 to 10:1, in the range of about 1:5 to 5:1, or in the range of about 1:1 to 8:1.

The films may be formed quickly, for example within less than about 30 minutes, less than about 20 minutes, less than about 10 minutes, or less than about 5 minutes, after the composition is applied to the skin.

Films according to the disclosure may be long-lasting. For example, once the composition is applied to the skin and a film is formed, the film may remain substantially intact on the skin for a period of at least about 12 hours, such as at least about 24 hours, at least about 48 hours, or at least about 72 hours.

The films may also be durable. For example, the film may not rub off, may not come off with sweat, or when the film is contacted by water, makeup, lotions, or other products that the user may wish to put on the skin.

### Methods

Methods of improving the appearance of the skin are also disclosed, said methods comprising applying a composition according to the disclosure onto the skin in order to form a film on the skin. Methods comprise tightening the skin, e.g. to get rid of eye bags.

It to be understood that, as used herein the terms “the,” “a,” or “an,” mean “at least one,” and should not be limited to “only one” unless explicitly indicated to the contrary. Thus, for example, reference to “a portion” includes examples having two or more such portions unless the context clearly indicates otherwise.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method that comprises A+B+C include embodiments where a method consists of A+B+C and embodiments where a method consists essentially of A+B+C. As described, the phrase “at least one of A, B, and C” is intended to include “at least one A or at least one B or at least one C,” and is also intended to include “at least one A and at least one B and at least one C.”

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints.

It is understood that when an amount of a component is given, it is intended to signify the amount of the active material.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

The compositions and methods according to the present disclosure can comprise, consist of, or consist essentially of the elements and limitations described herein, as well as any additional or optional ingredients, components, or limitations described herein or otherwise known in the art.

It will be apparent to those skilled in the art that various modifications and variations can be made in the delivery system, composition and methods of the invention without departing from the spirit or scope of the invention. Thus, it is intended that the present invention cover the modifications and variations of this invention provided that they come within the scope of the appended claims and their equivalents.

### EXAMPLES

The following Examples are provided for illustrative purposes only, and are not intended to be limiting.

In each of the following examples, the amounts of components given are in terms of active material (AM).

### Dynamic Mechanical Analysis (DMA)

The determination of Young Modulus of the films for all Examples was as follows. The film was made by using a draw down bar at 8 mil to cast the solution on a Teflon plate and dried the film at 40° C. in an oven overnight. The DMA Q800FR from TA instruments was used to measure the stress-strain response of the dried film. The deformation was applied from 0% strain to 200% strain at a rate of 100% strain/min at 32° C. Then the Young Modulus of the film was determined from the slope of the stress-strain curve in the linear viscoelastic regime.

### Scanning Electron Microscope (SEM) Measurement

The film sample for SEM was made by using the same method as for DMA measurement. Subsequently, the film was cut into a 5×5 mm piece and loaded onto a stage with a double sided carbon tape. The sample was scanned with a Hitachi TM-1000 Tabletop SEM.

### Rheology Measurement

The rheology of sample solutions was measured by using Rheometer AR-G2 from TA instruments. The dynamic oscillation mode was used with the parallel plate of 20 mm diameter at a gap of 200 μm.

The strain sweep from 0.001% to 1000% at an oscillation frequency of 1 rad/s was applied to the sample at 32° C. The value of elastic modulus G′ and viscous modulus G″ at 10% strain were recorded for each measured sample. The complex modulus G″ (consistency) and phase angle δ collected at 10% strain (in linear viscoelastic regime) were calculated from the elastic modulus G′ and viscous modulus G″ by the following equations:

$${G}^{*} = \sqrt{{G}^{′ 2} + {G}^{″ 2}}$$

$$δ = arctan ( \frac{{G}^{″}}{{G}^{′}} )$$

### Haze and Transparency—BYK Haze-Guard

The film was made by using a draw down bar at 8 mil to cast the solution on a transparent plastic film and dried on bench for 3 hours. The BYK Haze-Guard instrument was used to measure the transparency and the haze of the film.

### Gloss—BYK Glossmeter

The film was made by using a draw down bar at 8 mil to cast the solution on a transparent plastic film and dried on bench for 3 hours. The BYK Glossmeter was used to measure the gloss and matteness of the film.

### Film Permeability

The film was made by using a draw down bar at 8 mil to cast the solution on a Teflon plate and dried the film at 40° C. in an oven overnight. The film was peeled off and cut to 5×5 cm pieces. Each piece was used to cover the top of a scintillation vial filled with 2 mL water, and a piece of Parafilm was used to wrap the piece of film on the side. The weight of each vial was measured immediately as well as different time points. The water weight loss of different films was plotted to the different time points and the evaporation was calculated by fitting the evaporation curve with a linear function. The water vapor permeability of the film (P) is calculated with the followed equation, where (J) is the water vapor permeation flux; (l) is the thickness of the film and the (Δp) is the water vapor pressure difference between the space sealed by the film in the vial and the outside of the film, which is the ambient:

P=J/(Δp/l)

### Contact Angle Measurement

The film was made by using a draw down bar at 8 mil to cast the solution on a glass slide and dried on bench overnight. The contact angle of the film on the glass slide was measured by the Biolin Scientific Attension Tensiometer.

### Speed of Drying

The film was made by using a draw down bar at 8 mil to cast the solution on a transparent plastic film and weighed regularly during a period of one hour.

### Internal Constraint

A measured volume of formula is deposited and spread onto the nitrile band using a spatula or glass rod and let dry for a period of one hour. As the film shrinks upon drying, the surface of the nitrile band is measured by image analysis.

### Transparency, Homogenizing Power and Whitening Power—Colorimeter MINOLTA

The film was made by casting the solution on a transparent plastic film using a draw down bar (2 mil) and left to dry on the bench for 1 hour. The Minolta colorimeter was used to measure the L, a*, b* and Y of the film, and of a skin tone sheet reference and black and white reference, in order to calculate the transparency, homogenizing power, and whitening power of the films.

### Example 1

### Association of Thermoplastic Elastomer, Adhesive Polymer, and Filler

A thermoplastic elastomer, Kraton (25%), was dispersed in isoparaffin oil with a mechanical stirrer and heated to 90° C. Stirring continued at 90° C. for 1-2 hours until all Kraton polymer was dissolved and the polymer solution became clear. The desired amounts of oil dispersion (49% in isododecane), silica silylate and dimethicone crosspolymer were added into the Kraton/isoparaffin oil solution at the specified ratios in a plastic container, and the solution was mixed with a high speed mixer at 2500 rpm/min for 5 minutes. The final solution was kept at room temperature and sealed to avoid the evaporation of solvents.

The following Table 1 shows inventive formula prepared according to the disclosure

TABLE 1

| | Ex. 2a | Ex. 2b | Ex. 2c | Ex. 3d | Ex. 3e | Ex. 3f |
|---|---|---|---|---|---|---|
| Ratio-Kraton (AM):OD | 5 | 1 | 0.25 | 2 | 4 | 8 |
| (AM) | | | | | | |
| HYDROGENATED | 20.1% | 12.1% | 4.8% | 11.0% | 11.0% | 11.0% |
| STYRENE/BUTADIENE | | | | | | |
| COPOLYMER | | | | | | |
| OIL DISPERSION | 4.1% | 12.1% | 19.4% | 5.5% | 2.8% | 1.4% |
| SILICA SILYLATE | 3.0% | 3.0% | 3.0% | 3.0% | 3.0% | 3.0% |
| ISODODECANE | 38.3% | 38.3% | 38.3% | 47.5% | 50.3% | 51.6% |
| C8-9 ISOPARAFFIN | 34.5% | 34.5% | 34.5% | 33.0% | 33.0% | 33.0% |
| Total | 100.0% | 100.0% | 100.0% | 100.0% | 100.0% | 100.0% |
| G*(10% strain) Pa | 3389.3 | 1772 | 386.9 | | | |
| δ (10% strain)° | 34.8 | 35 | 31.5 | | | |
| Young Modulus (Mpa) at | 13.7 | 29.8 | 78.2 | 38.2 | 29.9 | 20.9 |
| 32° C. | | | | | | |
| CONCLUSION ON FILM | Acceptable | Good | Good | Good | Good | Good |

### Example 2

### Method of Use

In the section below, we describe the before and after effects of the inventive formula after application under the eye area. The experiment was conducted as followed. The face was split in 2 halves. The left side of the face was used as a baseline in order to be able to compare the effect of the product which will be applied on the right side of the face. Before applying the skin tightening composition, periorbital edema eye bags were observed on both sides of the face. It was considered the time T=0. Then, the product was applied under the right eye bag area before bed time. The left under eye area was left bare as a control in order to be able to compare the effects of the product overnight. The effects are described in the following paragraph.

The efficacy of the product was examined at T=10 min after application of the product and before bed time. As well as, the next morning, at T=7 hrs, before and after removal of the composition described herein. After application of the inventive formula only under the right eye bag area, a skin-tightening film was formed after 10 minutes showing a difference between the right side and the left side. This time constituted the T=10 min. The next morning at T=7 hrs, the left bare under eye bag area was showing an increase of the edema eye bag compare to the same eye bag area observed at T=0. On the contrary, the skin-tightening observed at T=10 min on the right side was still observed after overnight application of the composition described herein. We demonstrated that the application of the inventive skin tightening film under the eye area before going to bed, tightened the under eye area, and prevented the fluid build-up during the night. Indeed, this fluid build-up is the cause of the morning puffy eyes. This is a unique attribute of the film due to its strength and durability.

Thus, we demonstrated the effects on the appearance of the under eye area with and without the inventive example. It was observed that the inventive composition provided dramatic results under the eye bag area compared to the under eye bag area without the composition. An instant improvement was noticeable in 10 minutes after the application and lasted overnight. After removal of the product, the tightening under the eye bag area stayed the same proving that the application of the skin-tightening composition overnight help to fight the fluid build-up during the night. The removal revealed a fresh look. On the other hand, the left bare under eye bag area showed that the periorbital puffiness worsen overnight. So, the inventive composition provided conclusive results and proved that the application of the skin-tightening composition described herein eliminate the need to wear a tightening product during the day.

What is claimed is:

1. A method for treating periorbital puffiness, said method comprising:
(1) forming a film under the eye area skin by applying a skin-tightening composition to the under eye area, the skin-tightening composition comprising:
a. at least one thermoplastic elastomer chosen from amorphous hydrocarbon block copolymers of styrene and monomers of hydrocarbon containing 2 to 5 carbon atoms and comprising one or two ethylenic unsaturations, and having a first T_{g} below about 0° C., and a second T_{g} greater than about 25° C.;
b. at least one adhesive film-forming polymer chosen from polymer particles of C₁-C₄ alkyl(methacrylate)polymer, stabilized in a non-aqueous dispersion; and
c. at least one filler,
wherein the Young Modulus of the film formed on the under eye area is greater than about 500 kPa;
wherein the fluid under the eye accumulates substantially less after overnight treatment;
wherein the skin-tightening composition is applied before bed time.

2. The method of claim 1, wherein the step of applying further comprises applying the skin-tightening composition under the eye area for a duration of at least about 7 hours.

3. The method of claim 1, wherein the at least one thermoplastic elastomer is present in the composition in an amount ranging from about 5% to about 25% by weight, relative to the total weight of the composition.

4. The method of claim 1, wherein the at least one adhesive polymer is chosen from polymer particles comprising about 80% to about 100%, by weight, of C₁-C₄ alkyl (meth)acrylate and of about 0% to about 20%, by weight, of ethylenically unsaturated acid monomer of C₁-C₄ alkyl(methacrylate) polymer in an oil dispersion.

5. The method of claim 1, wherein the polymer of the particles is chosen from:
polymers consisting of at one or more C1-C4 alkyl(methacrylate)polymer; and
polymers consisting essentially of a copolymer of C1-C4 (meth)acrylate and of (meth)acrylic acid or maleic anhydride.

6. The method of claim 1, wherein the C₁-C₄ alkyl(methacrylate)polymer is chosen from methyl(meth)acrylate, ethyl (meth)acrylate, n-propyl (meth)acrylate, isopropyl (meth)acrylate, n-butyl (meth)acrylate and tert-butyl (meth)acrylate polymers.

7. The method of claim 1, wherein the skin-tightening composition further comprises at least one additional component chosen from silicone elastomers, humectants, and water.

8. The method of claim 1, wherein the at least one thermoplastic elastomer, at least one adhesive polymer, and at least one filler are present in the skin-tightening composition in a combined amount of greater than about 10% by weight, relative to the weight of the composition.

9. The method of claim 1, wherein the ratio of thermoplastic elastomer:adhesive polymer in the skin-tightening composition is in the range of about 1:1 to 8:1.

10. The method of claim 1, wherein the skin-tightening composition has a consistency G* of greater than about 100 Pa (at 10% strain) and a phase angle below about 45°.
